(19) United States
(12) Patent Application Publication
(10) Pub. No.: US 2020/0115358 A1
(43) Pub. Date: Apr. 16, 2020

(54) ICARIIN AND ICARITIN DERIVATIVES

(71) Applicant: H. LEE MOFFITT CANCER CENTER AND RESEARCH INSTITUTE, INC., Tampa, FL (US)

(72) Inventors: Sheng Wei, Tampa, FL (US); Alan List, Tampa, FL (US); Nicholas Lawrence, Tampa, FL (US)

(21) Appl. No.: 16/083,547

(22) PCT Filed: Mar. 13, 2017
(86) PCT No.: PCT/US17/22030

§ 371 Date: Sep. 11, 2018

Related U.S. Application Data
(60) Provisional application No. 62/306,694, filed on Mar. 11, 2016.

(51) Int. Cl. C07D 311/30; A61K 31/352; A61K 45/06; C07D 493/04; A61K 47/54

(52) U.S. Cl. CPC C07D 311/30; A61K 31/352; A61K 47/545; C07D 493/04; A61K 45/06

(57) ABSTRACT

Disclosed are derivatives of icariin. Disclosed are compounds having Formula I-V as defined herein. Methods of using these compounds for the treatment of cancer and inflammation are also disclosed.

### CROSS REFERENCE TO RELATED APPLICATIONS

This application claims the benefit of priority to U.S. Provisional Application 62/306,694, filed Mar. 11, 2016, which is incorporated by reference herein in its entirety.

### BACKGROUND

Inflammation is a hallmark of cancer and promotes the development and progression of cancer as well as the invasion of the immune system by tumor cells. Inflammation-induced cancer can be attributed to myeloid-derived suppressor cells (MDSCs), which accumulate in tumor bearing hosts, particularly in the local tumor microenvironment. MDSCs, characterized as Gr1⁺CD11b⁺ in mice and HLA-DR⁻Lin⁻CD33⁺ in humans, were identified as the major immune creator of an immunosuppressive and tumorigenic microenvironment (Gabrilovich D I, Nagaraj S. Nat Rev Immunol. 2009; 9(3):162-74). In healthy individuals, these cells exist as immature myeloid cells (IMC) and are part of normal myelopoiesis as they can quickly differentiate into mature monocytes, DC and neutrophils. However, under certain pathological situations, including inflammation and cancer, these IMCs are activated and accumulate in local tissues where they act both as tumor promoting and immunosuppressive cells through the release of soluble angiogenic and suppressive factors, such as VEGF, TGFβ, IL-6, or IL-10. They can also directly suppress tumor-specific CD4⁺ and CD8⁺ T-cell responses and induce CD4⁺CD25⁺FOXP3⁺ regulatory T cells (T_{regs}). Moreover, they can also contribute directly to the pathogenesis of cancer and leukemia by preventing the maturation of bone marrow progenitor cells as well as modulating hematopoietic stem cell/progenitor cell development. Further, reactive oxygen and nitrogen species (ROS and RNS respectively) and active STAT3 are implicated in MDSC function and are closely associated with up-regulation of immunosuppressive cytokines and tumor promoting factors. Hence targeting MDSCs and their downstream effector mechanisms is essential to restore immune recognition of the tumor and inhibit cancer progression. However, there are currently no effective therapeutic strategies to contain them.

Human MDSCs are unique in lacking all lineage markers and are defined by only one key receptor, CD33; a well-known surface marker of immature myeloid cells. It represents a 67 kDa type 1 transmembrane sialo-glycoprotein also known as the prototypical member of a subset of Sialic acid-binding Ig super-family lectins (SIGLEC). This particular subgroup is known as the CD33-related SIGLECs (CD33-r Siglecs) where CD33 is functionally known as SIGLEC 3. In humans, there are nine SIGLECs related to CD33, including SIGLEC 3, 5 and 14, which share 50-99% homology. Notwithstanding this homology, each SIGLEC has a unique specificity for sialylated ligands, making it more probable that each protein mediates a distinct function. All SIGLECs have an amino-terminal variable V-set immunoglobulin domain that binds sialic acid and, although the sugar moiety they bind is known, their complete ligand is not known. Another characteristic property of CD33-r SIGLECs, including SIGLEC 3, is the presence of two conserved immune-receptor tyrosine-based inhibitory motifs (ITIM) in their cytoplasmic region. Engagement of SIGLEC 3 with anti-SIGLEC 3 antibody, or through its ligand, leads to the phosphorylation of these tyrosine motifs which recruit and activate Src homology-2 (SH2) domain-containing tyrosine phosphatases (SHP-1 and SHP-2) (Paul S P et al. Blood. 2000; 96(2):483-90). Classically, receptors with ITIM domains function to suppress activation or maturation signals that emanate from receptors associated with activating motifs (ITAMs) through the recruitment of tyrosine and inositol phosphatases.

Additional CD33-r SIGLECs were discovered that deliver an activating, rather than inhibitory, signal. These alternative receptors lack ITIMs and instead interact with DAP12 (a DNAX-activating protein of 12 kDa). This interaction occurs through a positively charged anionic residue located in the transmembrane domain of the receptor, which non-covalently binds to a negatively charged aspartic acid residue on DAP12. This adaptor molecule is an ITAM-bearing protein shared by the majority of NK activating receptors. Signaling through it leads to the activation of Syk protein tyrosine kinase, phosphoinositide 3-kinase (PI3K), and ERK/MAPK. DAP12 partners with activating receptors, including SIGLEC-14 in humans and SIGLEC-H in mice, and plays a role in myeloid development through their involvement in the maturation and differentiation of hematopoietic stem cell into monocytes as well as promotion of DC maturation and survival. Therefore, DAP12 can down-regulate MDSC function and increase population numbers by counteracting SIGLEC3-ITIM signaling and driving MDSC differentiation into mature cells.

Recently, SIGLEC3's endogenous ligand was identified. Using a SIGLEC3-IgG Fc chimeric fusion protein, mass spectrometry identified a protein to be S100A9. This is significant because S100A8 and S100A9 (also called myeloid-related protein (MRP)-8 and 14 or Calgranulin A and B, respectively) can be a potent inflammatory mediator of MDSC activation in tumor-bearers. Furthermore, it has been found that SIGLEC 3-expressing MDSCs isolated from MDS patients (Myelodysplastic syndrome, a premalignant disorder that transforms to AML (acute myeloid leukemia)) have a high capacity for disruption of normal hematopoiesis (Wei S, et al. ASH Annual Meeting Abstracts. 2009; 114(22):597).

S100A8 and S100A9 (encoded by genes S100A8 and S100A9, respectively) are calcium-binding proteins expressed in myeloid cells during specific stages of differentiation and they are recognized as endogenous damage-associated molecular patterns (DAMPs). Working as a heterodimer (called Calprotectin), S100A8/A9 acts as an effective endogenous mediator to promote inflammation and MDSC activation. Furthermore, they are released at sites of ongoing inflammation leading to increased serum levels and correlating with the degree of inflammation. Using mice devoid of functional S100A8/A9, it has been established that both proteins can activate Toll like receptor-4 (TLR4) and hence are involved in TLR4-mediated signaling to promote inflammation. Up-regulation of S100A8/A9 in MDSCs can play a role in inhibition of DC and macrophage differentiation and can induce accumulation of MDSCs that can contribute to cancer development and tumor spread. Not only can S100A8 and S100A9 be related to the in vivo increase in the number of MDSCs in tumor-bearing mice but they can also be related to the inhibitory effects on myeloid cell differentiation. This idea was supported by S100A9 knock out mice that presented normal myeloid cell differentiation and greatly reduced MDSCs. In contrast, MDSC accumulation was enhanced in S100A9 transgenic mice (Tg) with inhibition of macrophage and DC differentiation (Cheng P et al. J Exp Med. 2008; 205(10):2235-49).

Given the current lack of effective targeted therapies to MDSC in cancer, along with their role in other inflammation associated diseases, inhibitors of MDSC are desirable. The compounds, compositions and methods disclosed herein address these and other needs.

### SUMMARY

In accordance with the purposes of the disclosed compounds, compositions and methods, as embodied and broadly described herein, the disclosed subject matter relates to compounds, compositions and methods of making and using the compositions. In more specific aspects, the disclosed subject matter relates to compounds that are derivatives of Icariin and Icaritin, methods of using the compounds, and compositions comprising the compounds. In certain aspects, the disclosed subject matter relates to compounds having the chemical structure shown in Formulas I-V, as defined herein. In still further aspects, the disclosed subject matter relates to methods for treating precancerous syndromes in a subject. For example, disclosed herein are methods whereby an effective amount of a compound or composition disclosed herein is administered to a subject having a precancerous syndrome, for example myelodisplastic syndrome, and who is in need of treatment thereof.

Additional advantages will be set forth in part in part in the description that follows and the Figures, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1: ICTA reduces levels of a-caspase-1, NLRP3, and colocalization of a-caspase-1/NLRP3 in cells treated with rhS100A9. Representative micrographs (1890× magnification) depicting inflammasome formation in U937 cells following 24 hour treatment with vehicle or 5 μg/mL rhS100A9 alone or with ICTA (20 μg/mL). DAPI (first column), a-caspase-1 (second column), NLRP3 (third column); merged image shows formation of inflammasome complexes (fourth column)

FIGS. 2A-2C: ICTA reduces levels of a-caspase-1, NLRP3, and colocalization of a-caspase-1/NLRP3 in cells treated with rhS100A9. Quantitative analysis of confocal images for (FIG. 2A) a-caspase-1, (FIG. 2B) NLRP3, and (FIG. 2C) colocalization. Error bars: SE, *p<0.05, **p<0.01, ***p<0.001.

FIGS. 3A-3D: In vivo inflammasome inhibition with ICTA improves hematopoiesis in S100A9Tg mice. At six months of age, S100A9Tg transgenic mice were treated every other day with 50 mg/kg of the inflammasome inhibitor ICTA by oral gavage for a total of eight weeks. Shown are changes in (FIG. 3A) hemoglobin, (FIG. 3B) white blood cells (WBC), (FIG. 3C) red blood cells (RBC) and (FIG. 3D) platelet counts in WT (n=4) and S100A9Tg (n=5) versus S100A9Tg mice treated with ICTA (n=5). Error bars: SE, *p<0.05, **p<0.01

FIG. 4: NLRP3 activation is reduced in bone marrow (BM) cells from ICTA-treated S100A9Tg transgenic mice. Representative micrograph (2520× magnification, 7.5 μm scale) depicting inflammasome formation in BM cells harvested from untreated S100A9Tg mice or mice treated with ICTA by oral gavage for a total of eight weeks. DAPI (first column), a-caspase-1 (second column), and NLRP3 (third column); merged images show inflammasome formation (fourth column).

FIG. 5: Nuclear β-catenin levels are reduced following in vivo treatment with ICTA. Representative micrographs (2520× magnification, 7.5 μm scale) of β-catenin expression in WT (n=5), S100A9Tg (n=5) and S100A9Tg that were treated with ICTA (n=5) by oral gavage for a total of eight weeks. DAPI (first column), β-catenin (second column); merged images show nuclear β-catenin localization (third column).

FIGS. 6A-6E: Wnt/β-catenin target gene expression is reduced in MDS BM-MNC (n=4) treated for 48 hours with ICTA. The following Wnt/β-catenin target genes were analyzed: (FIG. 6A) Cd44, (FIG. 6B) Ccnd1, (FIG. 6C) Ccne, (FIG. 6D) Cdk4, and (FIG. 6E) Cdk6.

FIGS. 7A-7B: ICTA reduces ASC polymerization. Representative density plot of inflammasome formation based on ASC oligomerization in (FIG. 7A) S34F control cells or (FIG. 7B) S34F cells treated with 10 μM ICTA.

FIG. 8: ICTA restores colony-forming capacity in U2AF1-S34F mutant cells. Colony forming capacity assessed in WT, S34F, and S34F cells treated with increasing concentrations of ICTA (0.01-10 μM). The mean number of colonies is representative of four replicates per condition. Error bars: SE, *p<0.05, **p<0.01.

FIG. 9: ICTA restores colony-forming capacity in SF3B1-K700E mutant BM cells. Colony forming capacity was assessed in WT, K700E or K700E cells treated with increasing concentrations of ICTA (0.1-10 μM). Mean number of BFU-E colonies is representative of BM cells isolated from four mice per condition, and four replicates per mouse. Error bars: SE, *p<0.05, **p<0.01, ***p<0.001.

### DETAILED DESCRIPTION

The compounds, compositions and methods described herein may be understood more readily by reference to the following detailed description of specific aspects of the disclosed subject matter and the Examples and Figures included therein.

Before the present compounds, compositions and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “treat” or other forms of the word, such as “treated” or “treatment,” is meant to administer a composition or to perform a method in order to reduce, prevent, inhibit, or eliminate a particular characteristic or event (e.g., tumor growth or survival). The term “control” is used synonymously with the term “treat.”

The term “anticancer” refers to the ability to treat or control cellular proliferation and/or tumor growth at any concentration.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, for example 1 to 3, 1 to 4, 1 to 5, 1 to 6, 1 to 7, 1 to 8, 1 to 9, 1 to 10, or 1 to 15 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms, for example, 2 to 5, 2 to 10, 2 to 15, or 2 to 20 carbon atoms, with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms, for example 2 to 5, 2 to 10, 2 to 15, or 2 to 20 carbon atoms, with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “carbonyl as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. Throughout this specification “C(O)” or “CO” is a short hand notation for C═O.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Compounds

Icariin (ICA) is a flavonoid glycoside derived from epimedium plants. Epimedium plants, also known as horny goat weed in the west or as Yinyanghuo in the Chinese pharmacopeia, contain an abundance of flavonoid glycosides.

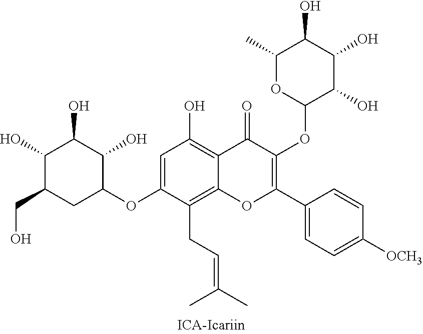 [SMILES: COc1ccc(-c2oc3c(CC=C(C)C)c(OC4C[C@@H](CO)[C@H](O)[C@@H](O)[C@@H]4O)cc(O)c3c(=O)c2OC2O[C@H](C)[C@@H](O)[C@H](O)[C@@H]2O)cc1]

Icariin and its deglycosolated derivative icaritin (3,5,7-trihydroxy-2-(4-methoxyphenyl)-8-(3-methyl-2-buten-1-yl)-4H-1-benzopyran-4-one), are thought to be responsible for the effects observed from herbal extracts of these plants, including enhanced anti-inflammatory and anti-tumorigenic activities.

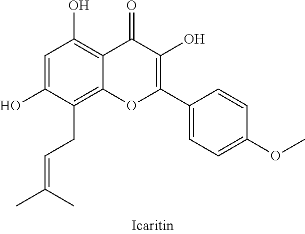 [SMILES: COc1ccc(-c2oc3c(CC=C(C)C)c(O)cc(O)c3c(=O)c2O)cc1]

ICA and a derivative 3,5,7-trihydroxy-4′-methoxy-8-(3-hydroxy-3-methylbutyl)-flavone) (ICT), were recently identified to effectively inhibit inflammatory responses associated with MDSCs (Zhou J et al. Int Immunopharmacol. 2011; 11(7):890-8; Wu J et al. Int Immunopharmacol. 2011; 12(1):74-9, which are incorporated by reference herein in their entireties for their teachings of ICA and ICT and their effect and use on MDSC and cancers).

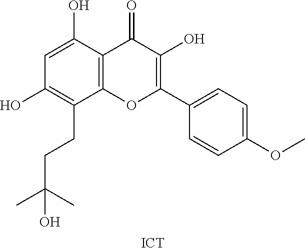 [SMILES: COc1ccc(-c2oc3c(CCC(C)(C)O)c(O)cc(O)c3c(=O)c2O)cc1 ; "ICT"]

These compounds disrupt the interaction of S100A8/A9 by reducing their expression, leading to a decrease in the number of peripheral and intratumoral MDSCs and inactivation of their activity, resulting in a reduced tumor burden.

Disclosed herein in one aspect are pharmaceutical compositions comprising derivatives of ICA, icaritin, and/or ICT with a pharmaceutical carrier, and optional anti-cancer and/or anti-inflammatory agent.

In a further aspect, disclosed herein are compounds that are derivatives of ICA and/or ICT. For example, disclosed herein are compounds having Formula I:

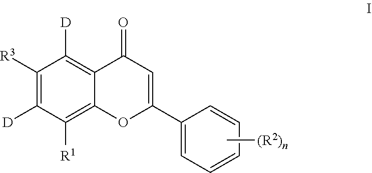

wherein,
each D, independent of the other, is chosen from H, OH, OR, and halogen;
R is alkyl or monoglucoside;
R¹ is chosen from hydrogen, halogen, hydroxyl, amino, thiol, thioalkyl, alkyl, alkenyl, alkynyl, haloalkyl, cycloalkyl, heterocycloalkyl, alkylaryl, aryl, alkylheteroaryl, and heteroaryl, any of which is optionally substituted with acetyl, alkyl, amino, amido, alkoxyl, alkylhydroxy, cycloalkyl, heterocycloalkyl, aryl, heteroaryl, carbonyl, halogen, hydroxyl, thiol, cyano, or nitro; or
R¹ and the adjacent D together form a fused heterocyclic ring which is optionally substituted with acetyl, alkyl, amino, amido, alkoxy, alkylhydroxy, cycloalkyl, heterocycloalkyl, aryl, heteroaryl, carbonyl, halogen, hydroxyl, thiol, cyano, nitro, sulfonyl, or sulfonlylamino;
each R², independent of any other, is chosen from hydrogen, hydroxyl, amino, thiol, nitro, cyano, sulfonyl, and an alkoxyl, thioalkyl, alkyl, alkenyl, alkynyl, haloalkyl, cycloalkyl, heterocycloalkyl, alkylaryl, aryl, alkylheteroaryl, or heteroaryl, any of which is optionally substituted with acetyl, alkyl, amino, amido, alkoxy, alkylhydroxy, cycloalkyl, heterocycloalkyl, aryl, heteroaryl, carbonyl, halogen, hydroxyl, thiol, cyano, nitro, sulfonyl, or sulfonlylamino;
n is 0, 1, 2, 3, 4 or 5;
R³ is chosen from hydrogen, alkyl, alkenyl, alkynyl, haloalkyl, cycloalkyl, heterocycloalkyl, alkylaryl, aryl, alkylheteroaryl, and heteroaryl, any of which is optionally substituted with acetyl, alkyl, amino, amido, alkoxy, alkylhydroxy, cycloalkyl, heterocycloalkyl, aryl, heteroaryl, carbonyl, halogen, hydroxyl, thiol, cyano, nitro, sulfonyl, or sulfonlylamino
or a pharmaceutically acceptable salt or prodrug thereof.

In certain examples, each D, independent of the other, is chosen from H, OH, OR, and halogen. In other specific examples, one D is H. In other examples, both D's are H. In still other examples, one D is OH. In other examples, both D's are OH. In yet further examples, one D is OR. In still other examples, both D's are OR. In still further examples, one D is OH and the other is OCH₃. In other examples, both D′s are OCH_{3.}
In certain examples, R¹ is chosen from alkyl, alkenyl, or alkoxyl, optionally substituted with with acetyl, alkyl, amino, amido, alkoxy, alkylhydroxy, cycloalkyl, heterocycloalkyl, aryl, heteroaryl, carbonyl, halogen, and hydroxyl. The alkyl or alkenyl can be from C₁ to C₂₄, more specifically, from C₁ to C₁₂, more specifically, from C₁ to C₈, such as from C₃ to C₆ in length. In other examples, R¹ is hydrogen and R³ is alkyl or alkenyl, which is optionally substituted with acetyl, alkyl, amino, amido, alkoxy, alkylhydroxy, cycloalkyl, heterocycloalkyl, aryl, heteroaryl, carbonyl, halogen, hydroxyl, thiol, cyano, nitro, sulfonyl, or sulfonlylamino; or a pharmaceutically acceptable salt or prodrug thereof,

In certain examples, R² is alkyl, alkenyl, or alkoxyl, optionally substituted with with acetyl, alkyl, amino, amido, alkoxy, alkylhydroxy, cycloalkyl, heterocycloalkyl, aryl, heteroaryl, carbonyl, halogen, hydroxyl, sulfonyl, or sulfonlylamino For example, R² can be methoxyl, ethoxyl, propyloxyl, methyl, ethyl, or propyl. In other examples R² is nitro.

In some specific examples of Formula I, where each D is OH, n is 1, R² is methoxyl, R³ is hydrogen, and R¹ is CH₂CH₂R⁴, the compounds have Formula II:

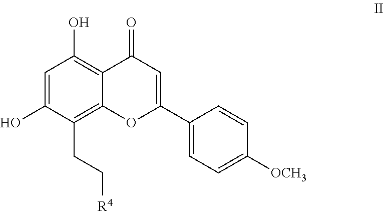

wherein R⁴ is selected from hydrogen, halogen, hydroxyl, amino, methylene, alkyl, alkenyl, alkynyl, haloalkyl, cycloalkyl, heterocycloalkyl, alkylaryl, aryl, alkylheteroaryl, or heteroaryl, any of which is optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro;
or a pharmaceutically acceptable salt or prodrug thereof.

In some examples of Formula II, R⁴ is an alkyl group, optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro. In some specific examples R⁴ is ═CH₂. In some specific examples R⁴ is CH(CH₃)₂.

In some further examples, where each D is OCH₃, n is 1, R² is methoxyl, R³ is hydrogen, and R¹ is CH₂CH₂R⁴, the compounds have Formula III:

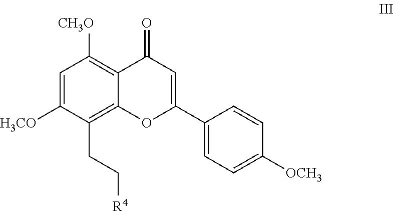

wherein R⁴ is selected from hydrogen, halogen, hydroxyl, amino, methylene, alkyl, alkenyl, alkynyl, haloalkyl, cycloalkyl, heterocycloalkyl, alkylaryl, aryl, alkylheteroaryl, and heteroaryl, any of which is optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro;
or a pharmaceutically acceptable salt or prodrug thereof.

In some examples of Formula III, R⁴ is an alkyl group, optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro. In some specific examples R⁴ is ═CH₂. In some specific examples R⁴ is CH(CH₃)₂.

Further examples are compounds of Formula IV:

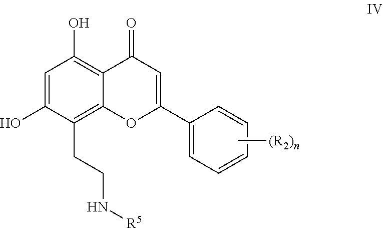

wherein R⁵ is selected from hydrogen, alkyl, alkenyl, alkynyl, haloalkyl, cycloalkyl, heterocycloalkyl, alkylaryl, aryl, alkylheteroaryl, and heteroaryl, any of which is optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro;
each R², independent of any other, is chosen from hydrogen, hydroxyl, alkoxyl, sulfonyl, amino, thiol, thioalkyl, alkyl, alkenyl, alkynyl, haloalkyl, cycloalkyl, heterocycloalkyl, alkylaryl, aryl, alkylheteroaryl, and heteroaryl, any of which is optionally substituted with acetyl, alkyl, amino, amido, alkoxy, alkylhydroxy, cycloalkyl, heterocycloalkyl, aryl, heteroaryl, carbonyl, halogen, hydroxyl, thiol, cyano, nitro, sulfonyl, or sulfonlylamino;
n is 0, 1, 2, 3, 4 or 5;
or a pharmaceutically acceptable salt or prodrug thereof.

In some further embodiments of Formula I, are compounds of Formula V:

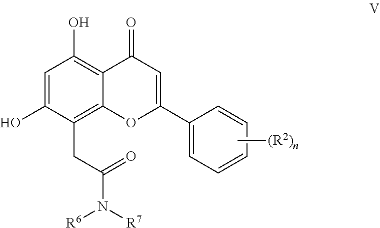

wherein R⁶ and R⁷ are independently selected from alkyl, alkenyl, alkynyl, haloalkyl, cycloalkyl, heterocycloalkyl, alkylaryl, aryl, alkylheteroaryl, and heteroaryl, any of which is optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro;
each R², independent of any other, is chosen from hydrogen, hydroxyl, alkoxyl, sulfonyl, amino, thiol, thioalkyl, alkyl, alkenyl, alkynyl, haloalkyl, cycloalkyl, heterocycloalkyl, alkylaryl, aryl, alkylheteroaryl, and heteroaryl, any of which is optionally substituted with acetyl, alkyl, amino, amido, alkoxy, alkylhydroxy, cycloalkyl, heterocycloalkyl, aryl, heteroaryl, carbonyl, halogen, hydroxyl, thiol, cyano, nitro, sulfonyl, or sulfonlylamino;
n is 0, 1, 2, 3, 4 or 5;
or a pharmaceutically acceptable salt or prodrug thereof.

In some examples, a hydrogen of any of the hydroxyls of in compounds of Formula I-V can be replaced with a linker of from 1 to 30 atoms in length bonded to biotin.

Specific examples of compounds disclosed herein are shown in Table 1.

TABLE 1

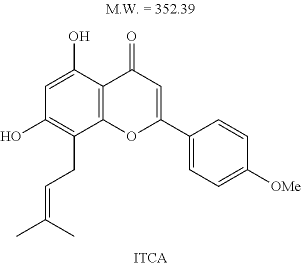 [SMILES: COc1ccc(-c2cc(=O)c3c(O)cc(O)c(CC=C(C)C)c3o2)cc1]

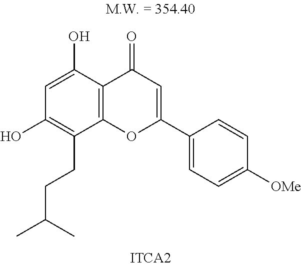 [SMILES: COc1ccc(-c2cc(=O)c3c(O)cc(O)c(CCC(C)C)c3o2)cc1]

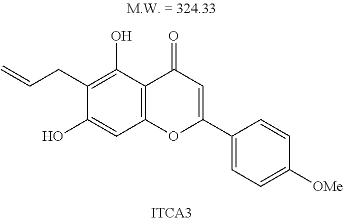 [SMILES: C=CCc1c(O)cc2oc(-c3ccc(OC)cc3)cc(=O)c2c1O]

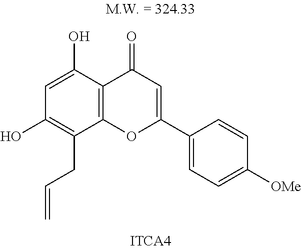 [SMILES: C=CCc1c(O)cc(O)c2c(=O)cc(-c3ccc(OC)cc3)oc12]

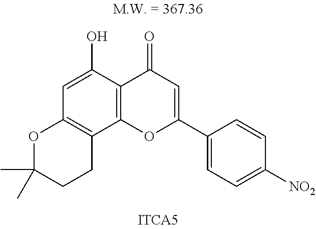 [SMILES: CC1(C)CCc2c(cc(O)c3c(=O)cc(-c4ccc([N+](=O)[O-])cc4)oc23)O1]

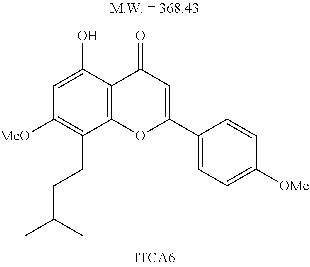 [SMILES: COc1ccc(-c2cc(=O)c3c(O)cc(OC)c(CCC(C)C)c3o2)cc1]

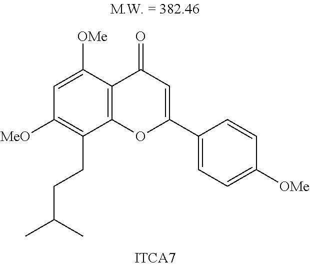 [SMILES: COc1ccc(-c2cc(=O)c3c(OC)cc(OC)c(CCC(C)C)c3o2)cc1]

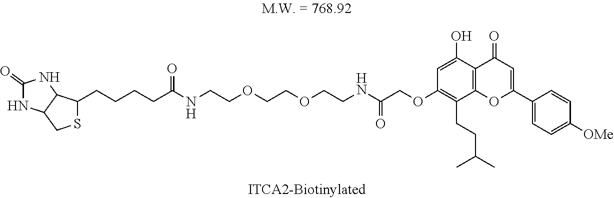 [SMILES: COc1ccc(-c2cc(=O)c3c(O)cc(OCC(=O)NCCOCCOCCNC(=O)CCCCC4SCC5NC(=O)NC54)c(CCC(C)C)c3o2)cc1]

Also disclosed herein are pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulphuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Compounds of Formulas I-V can be prepared beginning from Icaritin. For example the isopreneyl moiety on Icaritin can be oxidixed to an aldehyde, which can be reductively aminated to the amine or amide, or to the ester, which can be converted into the amide. Still further, the isoprenyl moiety can be oxidized to a carbonyl, which can be converted into a suitable leaving group for substitution reactions.

### Methods of Use

The compounds disclosed herein can be used to modulate the activation of MDSCs and alter the tumor microenvironment created by MDSCs. These compounds, and compositions containing them, can act through the down-regulation of S100A9/SIGLEC3 signaling, which is primordial to the function of MDSCs. The signaling event targeted by ICA/ICT and their derivatives disclosed herein can include direct or indirect inhibition of PDE5 and the activation of PP2A, which controls inflammatory mediators including the NO produced by MDSC. ICA/ICT and their derivatives disclosed herein can also be used to activate DAP12 to inhibit SIGLEC3-ITIM signaling and reduce the number of MDSC by driving their maturation. Treatment with ICA/ICT and its derivatives disclosed herein can reduce TNFα which mediates NO production. ICA/ICT and its derivatives disclosed herein can also down-regulate the levels of STAT3, which is a well-established transcription factor for MDSC expansion as well as production of suppressive cytokines (e.g. TGFβ), angiogenic factors (VEGF) and survival factors that benefit the establishment of the tumor. The receptor/ligand interactions that trigger these pathways are unclear but TLR4 has been favored as a major trigger in MDSC development leading to inflammation and cancer. TLR4 is a specialized receptor that can recognize not only exogenous but also endogenous danger signals, comprising pathogen-associated molecular patterns (PAMPs) as well as endogenous danger signals (DAMPs). S100A8/A9 is a potent DAMP released by cells that activate TLR4. The TLR4/MyD88/IRAK pathway can be critical for activation of numerous downstream effector pathways, including NF-κB, MAPK and STAT3. Deficiency of any of these markers can be associated with reduced tumor growth. It has been suggested that one of the most important tumor-promoting properties of these DAMP/receptor interactions is their ability to recruit MDSC to the tumor site. Therefore, in the context of cancer in a sterile environment, DAMPs can be responsible for setting off an inflammatory response that promotes tumor progression and local immune suppression.

Disclosed herein are thus methods of treating or preventing cancer in a subject, comprising administering to the subject an effective amount of a compound or composition as disclosed herein. Further provided herein are methods of treating a precancerous syndrome in a subject, comprising administering to the subject an effective amount of a compound or composition as disclosed herein. Examples of a precancerous syndromes, include, but are not limited to, myelodysplastic syndrome, essential throbocythaemia, myelofibrosis, monoclonal gammopathy of unknown significance (MGUS), polycythaemia vera, adenomatous polyps, familial adenomatous polyposis, hereditary non-polyposis colon cancer, submucous fibrosis, lichen planus, epidermolysis bullosa, discoid lupus erythematous, cervical dysplasia, cervical intraepithelial neoplasia, squamous intraepithelial lesion, epithelial hyperplasias, ductal carcinoma, and Paget's disease. Also provided are methods of sensitizing tumors to standard care therapy, comprising administering to the subject an effective amount of a compound or composition as disclosed herein.

Methods of killing a tumor cell are also provided herein. The methods comprise contacting a tumor cell with an effective amount of a compound or composition as disclosed herein. The methods can further include administering a second compound or composition (e.g., an anticancer agent) or administering an effective amount of ionizing radiation to the subject.

Methods of modifying a tumor microenvironment are also provided herein. The methods comprise contacting a tumor with an effective amount of a compound or composition as disclosed herein. Modification of the microenvironment can be characterized by a reduction in MDSCs as compared to control. The methods can further include administering a second compound or composition (e.g., an anticancer agent) or administering an effective amount of ionizing radiation to the subject.

Also provided herein are methods of radiotherapy of tumors, comprising contacting the tumor with an effective amount of a compound or composition as disclosed herein and irradiating the tumor with an effective amount of ionizing radiation. Methods of treating inflammation in a subject are further provided herein, the methods comprising administering to the subject an effective amount of a compound or composition as described herein. Optionally, the methods can further include administering a second compound or composition (e.g., an anti-inflammatory agent).

The disclosed subject matter also concerns methods for treating a subject having an oncological disorder or condition. In one embodiment, an effective amount of one or more compounds or compositions disclosed herein is administered to a subject having an oncological disorder and who is in need of treatment thereof. The disclosed methods can optionally include identifying a subject who is or can be in need of treatment of an oncological disorder. The subject can be a human or other mammal, such as a primate (monkey, chimpanzee, ape, etc.), dog, cat, cow, pig, horse, mouse or other animals having an oncological disorder. Means for administering and formulating compounds for administration to a subject are known in the art, examples of which are described herein. Oncological disorders include, but are not limited to, precancerous syndromes (such as MDS), cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include B cell cancers such as leukemia (acute lymphoblastic, acute myeloid, chronic lymphocytic, chronic myeloid, and other), lymphoma (Hodgkin's and non-Hodgkin's), and multiple myeloma.

Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

The disclosed subject matter also concerns methods for treating an infection and/or preventing sepsis in a patient in need thereof. Sepsis is caused by the immune system's response to a serious infection, most commonly bacteria, but also fungi, viruses, and parasites in the blood, urinary tract, lungs, skin, or other tissues.

The disclosed subject matter also concerns methods for treating a subject having an inflammatory and/or autoimmune disorder or condition. MDSC suppress immunity by perturbing both innate and adaptive immune responses. For example, MDSC indirectly affect T cell activation by suppressing CD4⁺ and CD8⁺ T cells by their uptake of arginine and high intracellular level of arginase that depletes their surroundings of arginine, an essential amino acid for T cell activation. In addition, MDSC-produced ROS and peroxynitrite inhibit CD8⁺ T cells by catalyzing the nitration of the TCR and thereby preventing T cell-peptide-MHC interactions. MDSC also perturb tumor immunity by skewing it toward a tumor-promoting type 2 phenotype. They do this by producing the type 2 cytokine IL-10 and by down-regulating macrophage production of the type 1 cytokine IL-12. This effect is amplified by macrophages that increase the MDSC production of IL-10. MDSC accumulation and activation are also identified with chronic inflammation. For example, proinflammatory cytokines IL-1β and IL-6 and the bioactive lipid PGE2 are known to induce MDSC.

Inflammatory and autoimmune disorders or conditions that can be treated by the compounds disclosed include, but are not limited to, systemic lupus erythematosus, Hashimoto's disease, rheumatoid arthritis, gouty arthritis, graft-versus-host disease, Sjögren's syndrome, pernicious anemia, Addison disease, scleroderma, Goodpasture's syndrome, inflammatory bowel diseases such as Crohn's disease, colitis, atypical colitis, chemical colitis; collagenous colitis, distal colitis, diversion colitis: fulminant colitis, indeterminate colitis, infectious colitis, ischemic colitis, lymphocytic colitis, microscopic colitis, gastroenteritis, Hirschsprung's disease, inflammatory digestive diseases, Morbus Crohn, non-chronic or chronic digestive diseases, non-chronic or chronic inflammatory digestive diseases; regional enteritis and ulcerative colitis, autoimmune hemolytic anemia, sterility, myasthenia gravis, multiple sclerosis, Basedow's disease, thrombopenia purpura, insulin-dependent diabetes mellitus, allergy; asthma, atopic disease; arteriosclerosis; myocarditis; cardiomyopathy; glomerular nephritis; hypoplastic anemia; rejection after organ transplantation and numerous malignancies of lung, prostate, liver, ovary, colon, cervix, lymphatic and breast tissues, psoriasis, acne vulgaris, asthma, autoimmune diseases, celiac disease, chronic prostatits, glomerulonephritis, inflammatory bowel diseases, pelvic inflammatory disease, reperfusion injury sarcoidosis, vasculitis, interstitial cystitis, type 1 hypersensitivities, systemic sclerosis, dermatomyositis, polymyositis, and inclusion body myositis.

In one embodiment, an effective amount of one or more compounds or compositions disclosed herein is administered to a subject having an inflammatory or autoimmune disorder and who is in need of treatment thereof. The disclosed methods can optionally include identifying a subject who is or can be in need of treatment of an inflammatory or autoimmune disorder. The subject can be a human or other mammal, such as a primate (monkey, chimpanzee, ape, etc.), dog, cat, cow, pig, horse, mouse or other animals having an inflammatory disorder. Means for administering and formulating compounds for administration to a subject are known in the art, examples of which are described herein.

Also disclosed is a method for treating a subject having a neurodegenerative disease or disorder. As used herein, “neurodegenerative disease” includes neurodegenerative disease associated with protein aggregation, also referred to as “protein aggregation disorders”, “protein conformation disorders”, or “proteinopathies”. Neurodegenerative disease associated with protein aggregation include diseases or disorders characterized by the formation of detrimental intracellular protein aggregates (e.g., inclusions in the cytosol or nucleus) or extracellular protein aggregates (e.g., plaques). “Detrimental protein aggregation” is the undesirable and harmful accumulation, oligomerization, fibrillization or aggregation, of two or more, hetero- or homomeric, proteins or peptides. A detrimental protein aggregate may be deposited in bodies, inclusions or plaques, the characteristics of which are often indicative of disease and contain disease-specific proteins. For example, superoxide dismutase-1 aggregates are associated with ALS, poly-Q aggregates are associated with Huntington's disease, and α-synuclein-containing Lewy bodies are associated with Parkinson's disease.

Neurological diseases are also associated with immune failure related to increasing levels of disease-causing factors that exceed the ability of the immune system to contain, or a situation in which immune function deteriorates or is suppressed concomitantly with disease progression, due to factors indirectly or directly related to the disease-causing entity. MDSCs can cause T-cell deficiency by suppressing effector T cell activity, thus promoting neurodegenerative disease associated with immune failure.

Representative examples of Protein Aggregation Disorders or Proteopathies include Protein Conformational Disorders, Alpha-Synucleinopathies, Polyglutamine Diseases, Serpinopathies, Tauopathies or other related disorders. Other examples of neurological diseases or include, but are not limited to, Amyotrophic Lateral Sclerosis (ALS), Huntington's Disease (HD), Parkinson's Disease (PD), Spinal Muscular Atrophy (SMA), Alzheimer's Disease (AD), diffuse Lewy body dementia (DLBD), multiple system atrophy (MSA), dystrophia myotonica, dentatorubro-pallidoluysian atrophy (DRPLA), Friedreich's ataxia, fragile X syndrome, fragile XE mental retardation, Machado-Joseph Disease (MJD or SCA3), spinobulbar muscular atrophy (also known as Kennedy's Disease), spinocerebellar ataxia type 1 (SCA1) gene, spinocerebellar ataxia type 2 (SCA2), spinocerebellar ataxia type 6 (SCA6), spinocerebellar ataxia type 7 (SCA7), spinocerebellar ataxia type 17 (SCA17), chronic liver diseases, familial encephalopathy with neuroserpin inclusion bodies (FENIB), Pick's disease, corticobasal degeneration (CBD), progressive supranuclear palsy (PSP), amyotrophic lateral sclerosis/parkinsonism dementia complex, Cataract, serpinopathies, haemolytic anemia, cystic fibrosis, Wilson's Disease, neurofibromatosis type 2, demyelinating peripheral neuropathies, retinitis pigmentosa, Marfan syndrome, emphysema, idiopathic pulmonary fibrosis, Argyophilic grain dementia, corticobasal degeneration, diffuse neurofibrillary tangles with calcification, frontotemporal dementia/parkinsonism linked to chromosome 17, Hallervorden-Spatz disease, Nieman-Pick disease type C, subacute sclerosing panencephalitis, cognitive disorders including dementia (associated with Alzheimer's disease, ischemia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced psychotic disorder; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); movement disorders, including akinesias and akinetic-rigid syndromes (including Parkinson's disease, drug-induced parkinsonism, postencephalitic parkinsonism, progressive supranuclear palsy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, and dyskinesias including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics), and dystonia (including generalized dystonia such as iodiopathic dystonia, drug-induced dystonia, symptomatic dystonia and paroxysmal dystonia, and focal dystonia such as blepharospasm, oromandibular dystonia, spasmodic dysphonia, spasmodic torticollis, axial dystonia, dystonic writer's cramp and hemiplegic dystonia)]; obesity, bulimia nervosa and compulsive eating disorders; pain including bone and joint pain (osteoarthritis), repetitive motion pain, dental pain, cancer pain, myofacial pain (muscular injury, fibromyalgia), perioperative pain (general surgery, gynecological), chronic pain, neuropathic pain, post-traumatic pain, trigeminal neuralgia, migraine and migraine headache; obesity or eating disorders associated with excessive food intake and complications associated therewith; attention-deficit/hyperactivity disorder; conduct disorder; mood disorders including depressive disorders, bipolar disorders, mood disorders due to a general medical condition, and substance-induced mood disorders; muscular spasms and disorders associated with muscular spasticity or weakness including tremors; urinary incontinence; amyotrophic lateral sclerosis; neuronal damage including ocular damage, retinopathy or macular degeneration of the eye, hearing loss or tinnitus; emesis, brain edema and sleep disorders including narcolepsy, and apoptosis of motor neuron cells. Illustrative examples of the neuropathic pain include diabetic polyneuropathy, entrapment neuropathy, phantom pain, thalamic pain after stroke, post-herpetic neuralgia, atypical facial neuralgia pain after tooth extraction and the like, spinal cord injury, trigeminal neuralgia and cancer pain resistant to narcotic analgesics such as morphine. The neuropathic pain includes the pain caused by either central or peripheral nerve damage. And it includes the pain caused by either mononeuropathy or polyneuropathy.

Further provided herein are methods of treating anemia of chronic disease (including cancer-related anemia) in a subject, comprising administering to the subject an effective amount of a compound or composition as disclosed herein.

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib. In other aspect, the disclosed compounds are coadministered with other HDAC inhibitors like ACY-1215, Tubacin, Tubastatin A, ST-3-06, OR ST-2-92.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

Data are expressed as means±standard error. Statistical analyses were carried out in Microsoft Excel using student's t-test, correlations using chi square for non-continuous variables and logistic regression for continuous variables were performed using IPSS software v22 (SPSS Inc., Chicago, Ill.), and *p values<0.05, **p<0.01, and ***p<0.01 were considered to be statistically significant.

### Example 1. In Vivo Inflammasome Inhibition Improves Hematopoiesis in S100A9Tg Mice

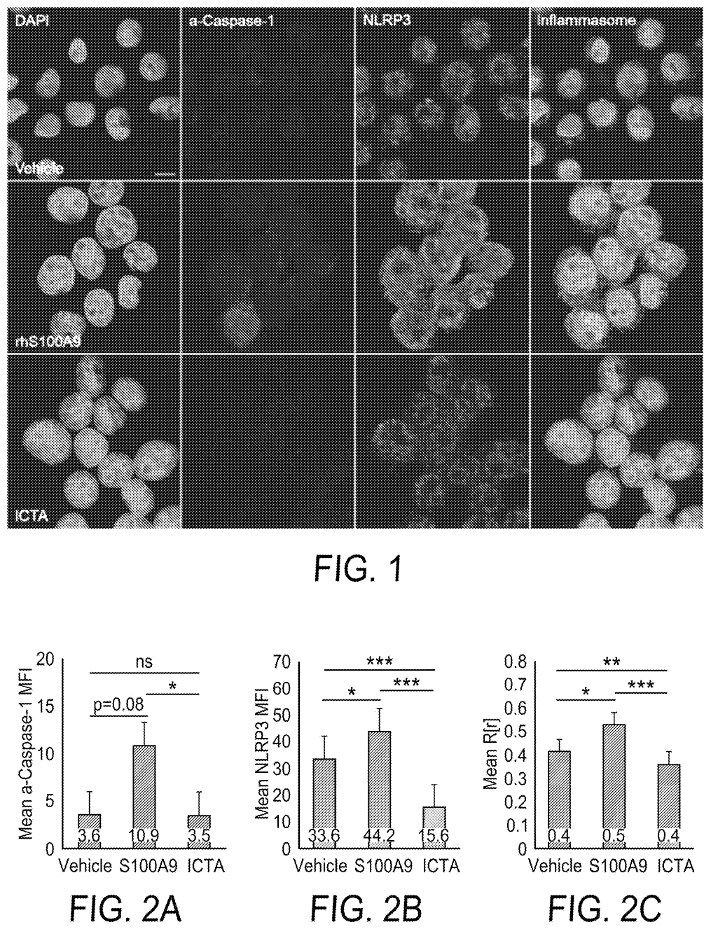

To test if in vivo inflammasome inhibition improves hematopoiesis in S100A9Tg mice analogous to human myelodysplastic syndrome (MDS), aged S100A9Tg mice were treated with ICTA, an Icariin derivative that inhibits NLRP3 inflammasome activation every other day for eight weeks. FIGS. 1 and 2A-2C show the reduction in a-caspase-1, NLRP3, and colocalization by ICTA treatment in U937 cells treated with rhS100A9.

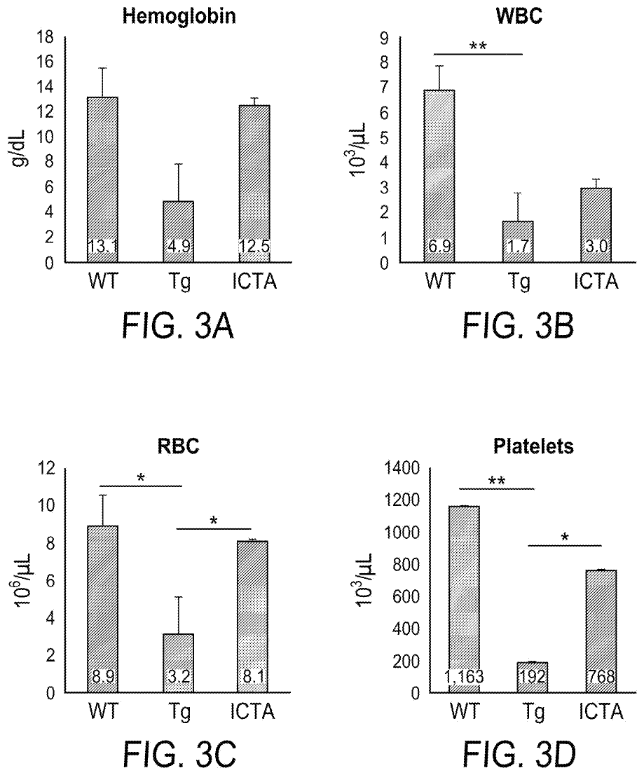

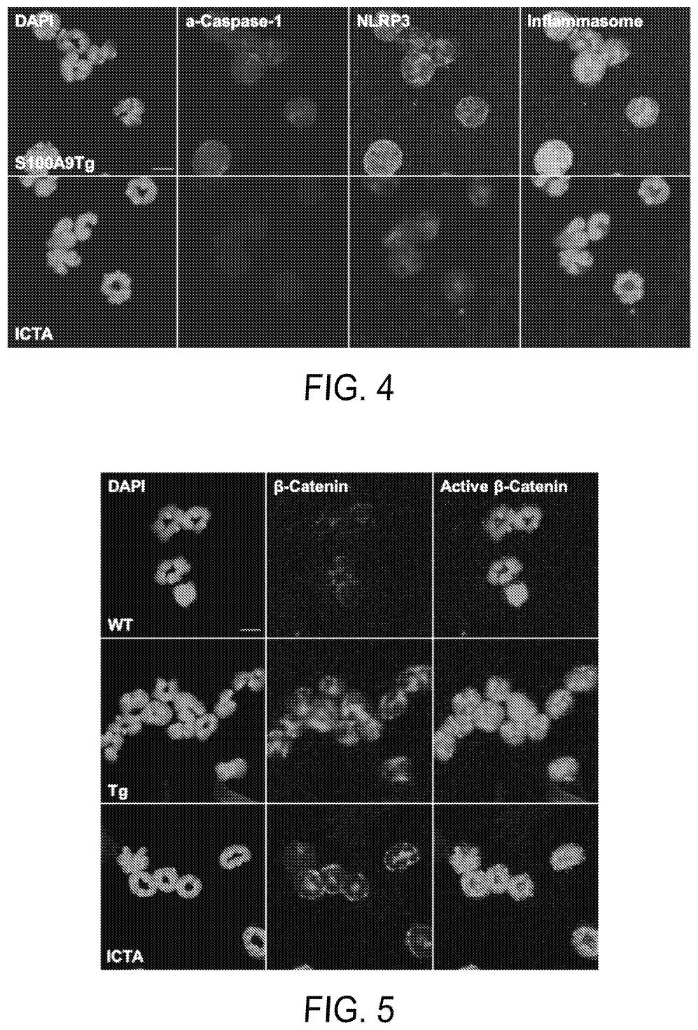

ICTA treated transgenic mice showed marked improvement in peripheral blood counts, including increased hemoglobin, leukocyte count (white blood cells), red blood cells and platelet counts (FIGS. 3A-3D), indicating restored effective hematopoiesis. Moreover, NLRP3 activation was reduced in BM cells from ICTA-treated transgenic animals (FIG. 4). Thus, pyroptosis is a principal mechanism driving HSPC cell death and MDS in S100A9Tg mice.

### Example 2. ICTA Reduces Levels of β-Catenin and Target Gene Expression

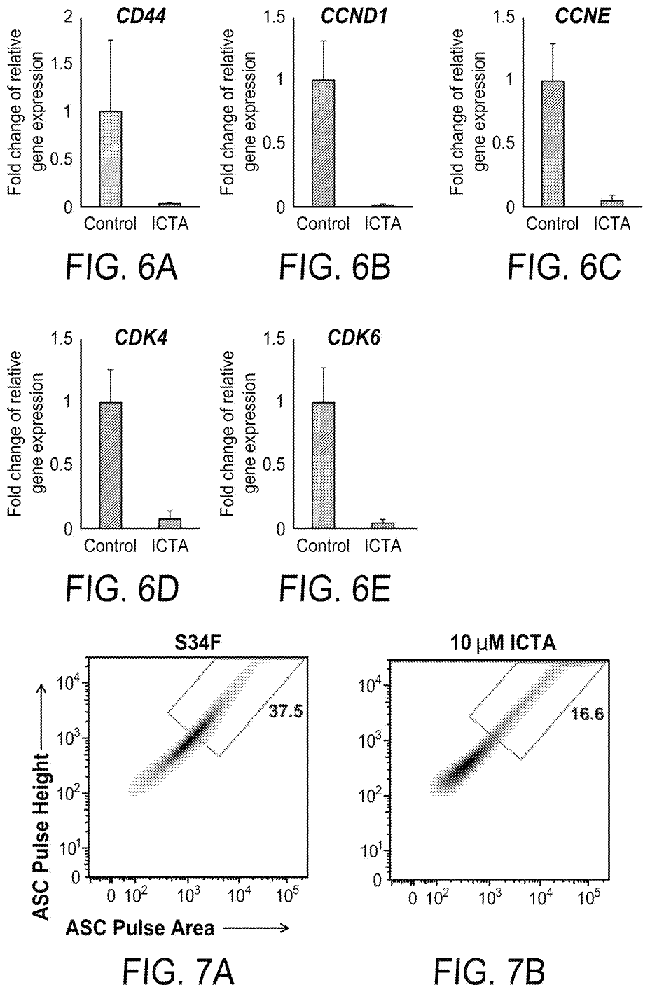

A significant increase in nuclear β-catenin in S100A9Tg-derived BM cells versus WT BM cells was observed, and levels of nuclear β-catenin were reduced following in vivo treatment with ICTA (FIG. 5). Similarly, treatment of MDS bone marrow-mononuclear cells (BM-MNCs) with ICTA suppressed nuclear β-catenin as well as target gene expression (FIGS. 6A-6E). Thus, S100A9-directed activation of β-catenin is a hallmark of myelodysplastic syndrome (MDS).

### Example 3. ICTA Reduces ASC Polymerization and Restores Colony-Forming capacity U2AF1-S34F Mutant Cells

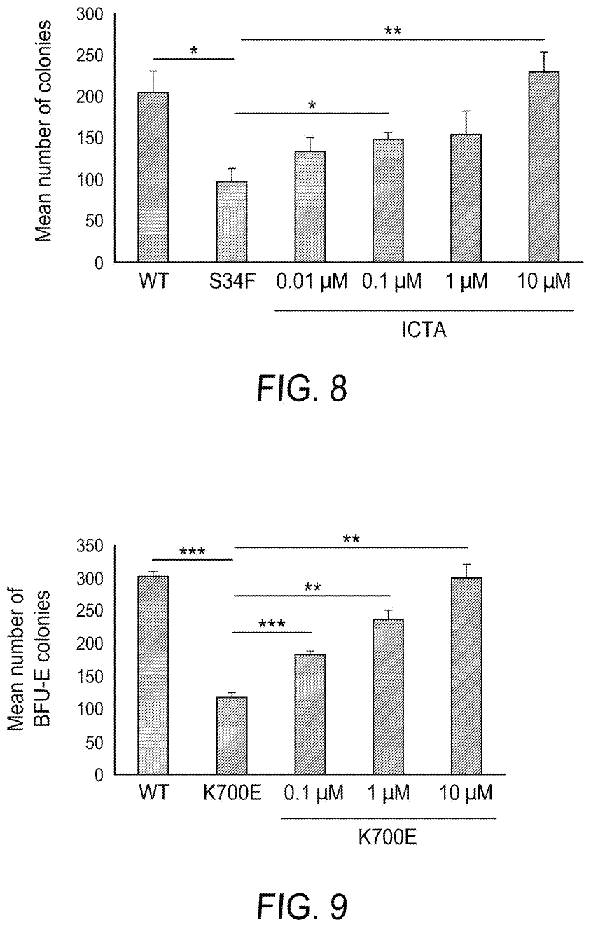

Treatment of the U2AF1-S34F mutant cells (cells contain a mutation in the U2AF splicing factor) with the NLRP3 inflammasome inhibitor ICTA suppressed inflammasome activation, as evidenced by a reduction in ASC polymerization, and restored colony-forming capacity to that of WT cells (FIGS. 7A, 7B, and 8). Thus, the reduced survival of cells harboring the MDS splicing mutation is driven by NLRP3 inflammasome-directed pyroptosis, while β-catenin activation may support propagation of the clone.

### Example 4. ICTA Restores Colony Forming Capacity in SF3B1-K700E Mutant BM Cells

Bone marrow (BM) cells were harvested from SF3B1-K700E conditional knock-in mice (n=3) which contain a mutation in the splicing factor SF3B1. These knock in mice display a myelodysplastic syndrome (MDS) phenotype (Obeng E. A, et al. Blood. 2014 124(6):828-30). Pharmacologic inhibition of the NLRP3 inflammasome using ICTA in SF3B1-K700E mutant BM cells restored colony forming capacity, illustrating the importance of inflammasome activation in the attrition of mutant cells (FIG. 9).

### Methods

MDS patient specimens. MDS patients consented on The University of South Florida Institutional Review Board approved protocols were recruited from the Malignant Hematology Clinic at H. Lee Moffitt Cancer Center & Research Institute, and the Eastern Cooperative Oncology Group (ECOG) E2905 trial (NCT00843882). Pathologic subtype of MDS was reported according to World Health Organization (WHO) criteria and prognostic risk assigned according to the International Prognostic Scoring System (IPSS). Patients were segregated as lower (Low, Intermediate-1) and higher risk (Intermediate-2, High) MDS. Bone marrow mononuclear cells (BM-MNC) were isolated from heparinized bone marrow aspirates using Ficoll-Hypaque Plus gradient centrifugation (GE Healthcare).

Mice. S100A9Tg mice were used for animal studies (Chen X, et al. J Clin Invest. 2013 123(11):4595-611). WT FVB/NJ mice were purchased from Jackson Laboratories (Bar Harbor, Me.). Heparinized BM cells were isolated from tibias and femurs of male and female mice.

Reagents and cells. U937 cells were grown in RPMI1640 supplemented with 10% FBS. TF-1 U2AF1 mutant and mock WT cells were cultured in RPMI1640 supplemented with 10% FBS and 2 ng/mL recombinant human GM-CSF. Cells were maintained at 37° C. under 5% CO₂. Normal, heparinized BM aspirates were purchased from Lonza Walkersville or AllCells, LLC. Normal and MDS bone marrow mononuclear cells (BM-MNC) were isolated from heparinized bone marrow aspirates using Ficoll-Hypaque Plus gradient centrifugation (GE Healthcare). Recombinant human S100A9 and the CD33/Siglec 3 chimeric fusion protein were generated as previously described (Chen X, et al. J Clin Invest. 2013 123(11):4595-611). Active caspase-1 and caspase-3/7 were detected using FAM-FLICA™ Caspase-1 and Caspase-3/7 activity kits, (ImmunoChemistry Technologies). NLRP1 antibodies were purchased from Santa Cruz Biotechnology, NLRP3 antibodies from Abcam, and β-catenin antibodies from BD Biosciences. Caspase-1 antibodies were purchased from Cell Signaling Technology, Inc. (#3866 and #14715, respectively).

Pyroptosis flow cytometry panel. For human samples, treated and untreated BM-MNC were incubated overnight in IMDM, supplemented with 10% autologous BM plasma. Subsequently, cells were harvested, washed twice in 1×PBS, and stained with LIVE/DEAD Violet fluorescent reactive dye according to the manufacturer's protocol (Life Technologies). Cells were resuspended in 1×PBS with 2% BSA, and incubated at room temperature for 15 minutes to block non-specific binding. After washing, cells were stained with 30×FAM-FLICA® Caspase-1 and Caspase-3/7 solution at a ratio of 1:30 for 2 hours at 37° C. Cells were washed and stained for cell surface receptors using CD38:PE-CF594, CD33:BV711, CD34:APC (BD Biosciences), and CD71:PE-Cyanine7 (eBioscience). All antibodies were diluted 1:20, and cells were stained for 30 minutes at 4° C. Cells were washed, resuspended in 1×binding buffer, and stained with Annexin-V:Cy5.5 at a dilution of 1:20 for 15 minutes at room temperature (BD Biosciences). 1×binding buffer was added to a final volume of 400 μL. Sample acquisitions were carried out using a BD LSR II flow cytometer and FACSDiva software (BD Biosciences). Calibration of the flow cytometer was carried out prior to each experiment using Rainbow Mid-Range Fluorescent Particles (BD Biosciences). To establish fluorescence compensation settings, ArC Amine Reactive Compensation Beads were used for LIVE/DEAD Violet staining (Life Technologies), and BD CompBead Plus Anti-Mouse Ig κ/Negative Control (BSA) Compensation Plus Particles were used for surface receptor conjugates (BD Biosciences). Data were analyzed using FlowJo 9.7.5 software (FlowJo, LLC, Ashland, Oreg.).

S100A9 flow cytometry experiments in U937 cells. Monocytic U937 cells were treated with the indicated concentrations of rhS100A9 for 24 hours, or with 5 μg/mL rhS100A9 for the indicated time points. Subsequently, cells were stained with 30×FAM-FLICA® Caspase-1 solution at a ratio of 1:30 for 2 hours at 37° C. Cells were washed, resuspended in 1×binding buffer, and stained with Annexin-V:PE at a dilution of 1:30 for 15 minutes at room temperature. 1×binding buffer was added to a final volume of 350 μL. Sample acquisitions were carried out using a BD FACSCalibur flow cytometer (BD Biosciences). Data were analyzed using FlowJo 9.7.5 software.

Intracellular S100A9 flow cytometry. BM-MNC were incubated overnight in IMDM, supplemented with 10% autologous BM plasma. The following day, cells were harvested and washed twice in 1×PBS. Cells were fixed with BD Cytofix Fixation Buffer at 37° C. for 10 minutes, and stored at −80° C. until staining. At the time of staining, cells were warmed to 37° C. in a water bath, spun down, and washed 1×with staining buffer. Pellets were resuspended in 1 mL of pre-warmed BD Permeabilization Buffer III, and incubated on ice for 30 minutes. Cells were washed twice with staining buffer. Following washing, cells were stained with S100A9:FITC (BioLegend), and cell surface receptors using CD38:PE-CF594, CD33:BV711, CD34:APC (BD Biosciences), and CD71:PE-Cyanine7 (eBioscience). All antibodies were diluted 1:20, and cells were stained for 30 minutes at 4° C. Cells were washed and resuspended in 400 μL staining buffer. Sample acquisitions were carried out using a BD LSR II flow cytometer and FACSDiva software (BD Biosciences).

Immunofluorescence confocal microscopy. Mouse BM cells were stained with 30×FAM-FLICA™ Caspase-1 solution at a ratio of 1:30 for 2 hours at 37° C. Cells were washed and cytospins were generated using a 5 minutes centrifugation at 450 rpm. Slides were fixed at 37° C. for 10 minutes using BD Cytofix Fixation Buffer (BD Biosciences), and subsequently washed using PBS. Cells were permeabilized with 0.1% Triton X-100/2% BSA in PBS for 15 minutes at room temperature. After washing with PBS, cells were blocked using 2% BSA in PBS for 30 minutes at room temperature, and washed again. Cells were incubated with the appropriate primary antibody overnight (1:400 for NLRP3, 1:20 for β-catenin) at 4° C. The next day, cells were washed with PBS and incubated with the appropriate secondary antibodies (1:500) for 1 hour at room temperature. After washing, cells were covered with ProLong Gold Antifade Reagent with DAPI prior to the addition of a coverslip (Life Technologies). Co-localization of a-caspase-1 with NLRP3 inflammasome complexes was assessed using a Leica TCS SP5 AOBS Laser Scanning Confocal microscope (Leica Microsystems). Analysis of the inflammasome images was performed with Definiens Developer 2.0 (Definiens AG). The software was used to segment cells based on brightness and size thresholds, followed by a watershed segmentation algorithm. Intensity values and Pearson's correlation coefficient were extracted from the segmented cells. For β-catenin image analysis, confocal images were imported into Definiens Tissue Studio v3.0, 64 Dual in .tif format. Cells were separated from background using the RGB thresholds. Nuclei were identified by setting thresholds in the DAPI channel. Typical cells averaged 60 microns. The red intensity (β-catenin) in the nucleus and cytoplasm was established by setting thresholds to low, medium and high in the red channel on a scale of 0-255 in the red channel

ASC staining to detect inflammasome formation by flow cytometry. Staining was carried out as described (Sester D. P, et al. J Immunol. 2015 194(1):455-62). Briefly, cell pellets were resuspended in 1 mL of prewarmed BD Permeabilization Buffer III, and incubated on ice for 30 minutes. Cells were washed 2× with staining buffer. Following washing, cells were stained with rabbit-anti-ASC primary antibodies at a 1:1500 dilution and incubated for 90 minutes. Cells were washed, stained with secondary antibodies at a dilution of 1:1500, and incubated for 45 minutes. Cells were washed, and sample acquisitions were carried out using a BD LSR II flow cytometer and FACSDiva software.

ASC speck detection. 400 μg of protein was aliquoted from BM plasma from normal donors and MDS patients, stained with rabbit-anti-ASC primary antibodies at a 1:1500 dilution and incubated for 90 minutes. Secondary antibodies were added at a dilution of 1:1500 and incubated for 45 minutes. Sample acquisitions were carried out using a BD FACSCalibur flow cytometer. Threshold for FSC, SSC and the secondary fluorochrome was set to zero to allow detection of specks.

Real-time quantitative PCR. RNA was isolated from BM-MNC using the RNeasy Mini Kit (Qiagen). cDNA was produced using the iScript cDNA Synthesis Kit (Bio-Rad). Sequences for primers can be found in Table 1. GAPDH mRNA was used for transcript normalization. cDNA was amplified using the iQ SYBR Green Supermix and the CFX96 Real-Time PCR Detection System (Bio-Rad). PCR conditions were as follows: 10 minutes at 95° C., followed by 40 cycles of amplification (15 seconds at 95° C. and 1 minute at 60° C.). Relative gene expression was calculated using the −2^{ΔΔCt} method.

TABLE 1
Primer sets used for qPCR.

| Gene | | |
|---|---|---|
| | Forward | |
| GAPDH | 5′-GAAGGTGAAGGTCGGACT-3′ | SEQ ID NO: 1 |
| | Reverse | |
| GAPDH | 5′-GAAGATGGTGATGGGATTTC-3′ | SEQ ID NO: 2 |

Colony formation assay. Four replicates of 350,000 BM-MNC/mL were resuspended in 10% autologous BM plasma and plated in MethoCult methylcellulose medium (Stemcell Technologies) supplemented with 1% v/v penicillin-streptomycin and 3 units/mL erythropoietin. CD33-IgG and MCC950 were added directly to the medium prior to plating. Colonies of BFU-E, CFU-GM, and CFU-GEMM were scored using an inverted light microscope fourteen days after plating. For U2AF1 assays, four replicates of 30,000 cells/mL were plated in medium supplemented with 1% v/v penicillin-streptomycin and increasing concentrations of ICTA. Colonies were counted seven days after plating. For SF3B1-K700E assays, BM cells were isolated from four donors per condition and four replicates of 350,000 BM cells/mL were plated in MethoCult methylcellulose medium for murine cells with increasing concentrations of ICTA. Colonies were scored fourteen days after plating.

ICTA mouse treatment studies. ICTA was synthesized by the Drug Discovery Core Facility at H. Lee Moffitt Cancer Center & Research Institute. Six month old transgenic mice (n=5) were dosed every other day with 50 mg/kg ICTA by oral gavage, for a total of eight weeks.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A compound having Formula I: 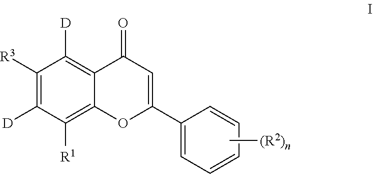
wherein,
each D, independent of the other, is chosen from H, OH, OR, and halogen;
R is alkyl or monoglucoside;
R¹ is chosen from hydrogen, halogen, hydroxyl, amino, thiol, thioalkyl, alkyl, alkenyl, alkynyl, haloalkyl, cycloalkyl, heterocycloalkyl, alkylaryl, aryl, alkylheteroaryl, and heteroaryl, any of which is optionally substituted with acetyl, alkyl, amino, amido, alkoxyl, alkylhydroxy, cycloalkyl, heterocycloalkyl, aryl, heteroaryl, carbonyl, halogen, hydroxyl, thiol, cyano, or nitro; or
R¹ and the adjacent D together form a fused heterocyclic ring which is optionally substituted with acetyl, alkyl, amino, amido, alkoxy, alkylhydroxy, cycloalkyl, heterocycloalkyl, aryl, heteroaryl, carbonyl, halogen, hydroxyl, thiol, cyano, nitro, sulfonyl, or sulfonlylamino;
each R², independent of any other, is chosen from hydrogen, hydroxyl, amino, thiol, nitro, cyano, sulfonyl, and an alkoxyl, thioalkyl, alkyl, alkenyl, alkynyl, haloalkyl, cycloalkyl, heterocycloalkyl, alkylaryl, aryl, alkylheteroaryl, or heteroaryl, any of which is optionally substituted with acetyl, alkyl, amino, amido, alkoxy, alkylhydroxy, cycloalkyl, heterocycloalkyl, aryl, heteroaryl, carbonyl, halogen, hydroxyl, thiol, cyano, nitro, sulfonyl, or sulfonlylamino;
n is 0, 1, 2, 3, 4 or 5;
R³ is chosen from hydrogen, alkyl, alkenyl, alkynyl, haloalkyl, cycloalkyl, heterocycloalkyl, alkylaryl, aryl, alkylheteroaryl, and heteroaryl, any of which is optionally substituted with acetyl, alkyl, amino, amido, alkoxy, alkylhydroxy, cycloalkyl, heterocycloalkyl, aryl, heteroaryl, carbonyl, halogen, hydroxyl, thiol, cyano, nitro, sulfonyl, or sulfonlylamino
or a pharmaceutically acceptable salt or prodrug thereof.

2. The compound of claim 1, wherein each D is a hydroxyl group.

3. The compound of claim 1, wherein each D is a methoxyl group.

4. (canceled)

5. The compound of claim 1, wherein n is 1.

6. The compound of claim 1, wherein R² is a methoxy group.

7. The compound of claim 1, wherein R³ is hydrogen, alkyl, or alkenyl.

8. A compound having Formula II: 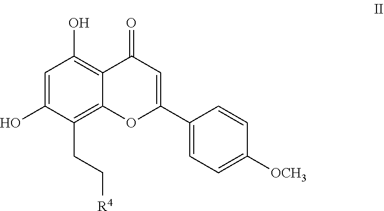
wherein R⁴ is selected from hydrogen, halogen, hydroxyl, amino, methylene, alkyl, alkenyl, alkynyl, haloalkyl, cycloalkyl, heterocycloalkyl, alkylaryl, aryl, alkylheteroaryl, and heteroaryl, any of which is optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro;
or a pharmaceutically acceptable salt or prodrug thereof.

9. The compound of claim 8, wherein R⁴ is an alkyl group, optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro.

10. The compound of claim 8, wherein R⁴ is ═CH₂.

11. The compound of claim 8, wherein R⁴ is CH(CH₃)₂.

12. A compound having Formula III: 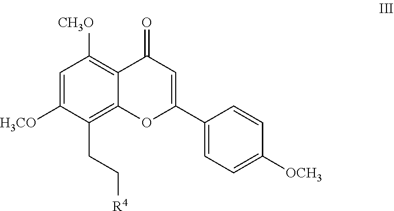
wherein R⁴ is selected from hydrogen, halogen, hydroxyl, amino, methylene, alkyl, alkenyl, alkynyl, haloalkyl, cycloalkyl, heterocycloalkyl, alkylaryl, aryl, alkylheteroaryl, and heteroaryl, any of which is optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro;
or a pharmaceutically acceptable salt or prodrug thereof.

13. The compound of claim 12, wherein R⁴ is an alkyl group, optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro.

14. The compound of claim 12, wherein R⁴ is ═CH₂.

15. The compound of claim 12, wherein R⁴ is CH(CH₃)₂.

16. A compound having Formula IV or Formula V: 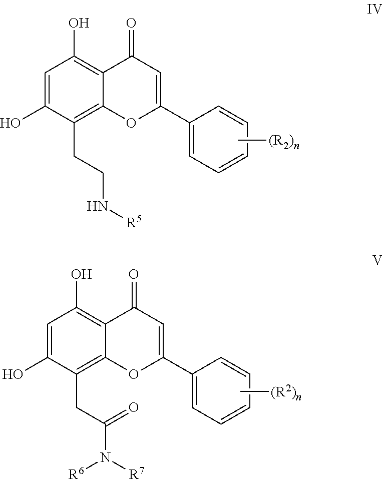
wherein R⁵ is selected from hydrogen, alkyl, alkenyl, alkynyl, haloalkyl, cycloalkyl, heterocycloalkyl, alkylaryl, aryl, alkylheteroaryl, and heteroaryl, any of which is optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro;
R⁶ and R⁷ are independently selected from alkyl, alkenyl, alkynyl, haloalkyl, cycloalkyl, heterocycloalkyl, alkylaryl, aryl, alkylheteroaryl, and heteroaryl, any of which is optionally substituted with carbonyl, alkyl, amino, amido, alkoxyl, alkylhydroxy, cycloalkyl, heterocycloalkyl, aryl, heteroaryl, carbonyl, halogen, hydroxyl, thiol, cyano, or nitro;
each R², independent of any other, is chosen from hydrogen, hydroxyl, alkoxyl, sulfonyl, amino, thiol, thioalkyl, alkyl, alkenyl, alkynyl, haloalkyl, cycloalkyl, heterocycloalkyl, alkylaryl, aryl, alkylheteroaryl, and heteroaryl, any of which is optionally substituted with acetyl, alkyl, amino, amido, alkoxy, alkylhydroxy, cycloalkyl, heterocycloalkyl, aryl, heteroaryl, carbonyl, halogen, hydroxyl, thiol, cyano, nitro, sulfonyl, or sulfonlylamino;
n is 0, 1, 2, 3, 4 or 5;
or a pharmaceutically acceptable salt or prodrug thereof.

17. (canceled)

18. A compound chosen from

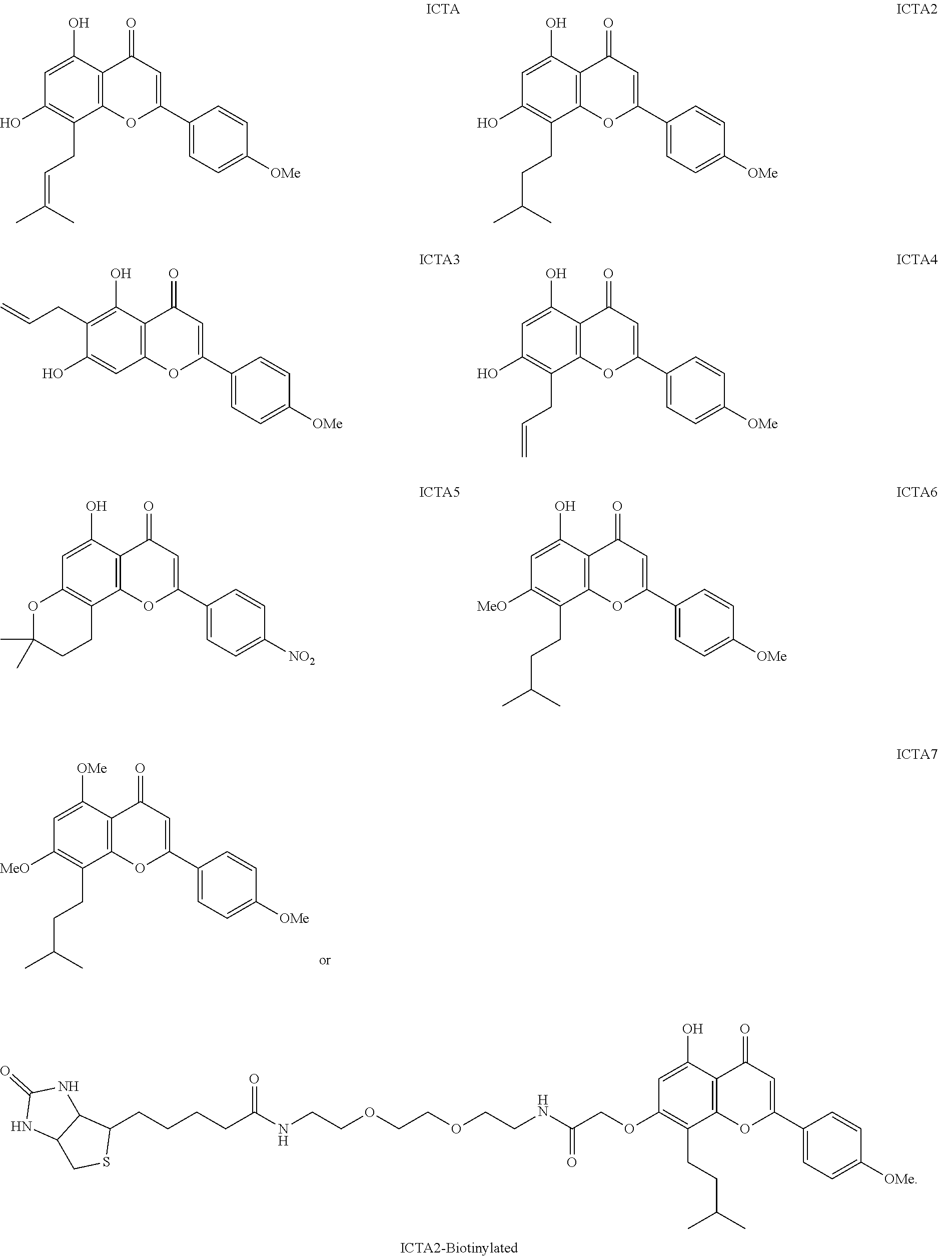

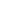

19. (canceled)

20. A pharmaceutical composition comprising a therapeutically effective amount of a compound of claim 1 and a pharmaceutical carrier and optional anticancer or anti-inflammatory agent.

21. A method of treating myelodysplastic syndrome comprising: administering to the subject a therapeutically effective amount of a compound of claim 1.

22. A method of killing a tumor cell, comprising contacting a tumor cell with an effective amount of a compound or composition of claim 1.


SEQUENCE LISTING (2 sequences)

SEQ ID NO: 1 - DNA, 18 bp, Artificial Sequence
  synthetic construct
gaaggtgaaggtcggact

SEQ ID NO: 2 - DNA, 20 bp, Artificial Sequence
  synthetic construct
gaagatggtgatgggatttc